(19) United States
(12) Patent Application Publication
(10) Pub. No.: US 2008/0287978 A1
(43) Pub. Date: Nov. 20, 2008

(54) MEDICAL MAPPING DEVICE

(76) Inventor: Charles B. Hickman, III, Ashland, KY (US)

Correspondence Address: ROBERT R. WATERS, ESQ.;WATERS LAW OFFICE, PLLC, 633 SEVENTH STREET, HUNTINGTON, WV, 25701, US

(21) Appl. No.: 11/751,012

(22) Filed: May 19, 2007

(51) Int. Cl. A61M 37/00

(52) U.S. Cl. 606/186; 604/244

(57) ABSTRACT

A medical tattooing device is disclosed. A needle and attached needle cap have a drive spring seated against them in a pre-loaded position so that the needle and needle cap need only be released to send the needle forward and create the tattoo. Anchoring tabs temporarily anchor the needle and needle cap. A push button is used to break the tabs and release the needle. One end of the device has a seal ink reservoir. Before applying the tattoo, a tab on the reservoir is broken off to open the reservoir to passage of the needle and flow of ink. The device is placed on the skin, the button pushed to break the tabs, and the needle passes through the reservoir to apply the tattoo. A rebound spring opposing the drive spring retracts the needle the desired amount.

### FIELD OF THE INVENTION

This invention relates generally to an improved device for placing a permanent though inconspicuous tattoo upon a person's body. The tattoo maps a point for repeated medical treatments such as radiation therapy and allows accurate application of the treatment without spending time relocating the precise location desired. One application for the device is in radiation oncology simulation, or R.O.S. Some embodiments of the device have their own ink reservoir and the device is disposable for sanitary purposes.

### BACKGROUND OF THE INVENTION

Many medical treatments consist of repeatedly applying treatment to the same area of the body over a period of time. A common type of illness that requires this is cancer. Radiation treatments are applied to an afflicted area over a period of time during a series of visits to the hospital or outpatient facility. With the constant improvement in medical technology, these radiation treatments are much more narrowly focused than they once were. This reduces the level of radiation dosage required and reduces the amount of directly affected tissue. However, the treatments require greater accuracy and repeatability. Small, permanent tattoos are used to assist this accuracy. The point of application for the treatment is mapped at the initial treatment, and the tattoo is applied for subsequent treatments. This approach to radiation treatment is frequently referred to as radiation oncology simulation, or R.O.S.

Tattoos themselves are indeed ancient. The basic procedure involves repeatedly perforating the skin with shallow punctures in the presence of ink, dye, or other source of pigment of the desired color. The ink enters the skin through the perforations and permanently colors the skin. The perforations may be created with a single needle or a cluster of needles held together. This procedure can be accomplished with the simplest of means, and the depth of the perforations need be through only the first three, or so, layers of skin. Shallow perforations not passing through the skin are adequate to create a tattoo and are actually preferred over deep perforations to prevent the flow of plasma. Plasma flow from the skin fades and smears the newly applied tattoo.

Modern methods of tattooing use tattooing machines which drive the needle or needles in reciprocating action to perforate the skin. If a cluster of needles is used, the needles are held together with a needle bar which in turn mounts into the tattooing apparatus. These needles are disposable in consideration of general affluence and awareness of communicable infection. The needles, however, are not necessarily the only part of the tattooing apparatus that may come into contact with a body or bodily fluids. For this reason, some apparatuses use a disposable guide surrounding the needle as well. More developed tattooing machines also have a means of delivering ink to the skin during the tattooing process. For these apparatuses, the needle, needle holder, and needle guide can connect with a driver mechanism and ink supply modularly and can then be thrown away after one use.

For the purpose of applying a medical tattoo, a full size tattooing machine is not necessary. The tattoo is usually quite small. The need is much more defined by simplicity of use, cost effectiveness, disposable sanitary methods, and painless application of the tattoo. Because of the smallness of the tattoo, it is not an uncommon practice to use ink from a common source. Small amounts of ink are drawn or dipped from this common source and applied to the skin where a tattoo will be applied. This repeated contact with the ink source conflicts with sanitary requirements. In addition to contamination from a common ink source, excessive amounts of ink may be introduced into or beneath the skin. A mark resulting from excessive ink, or too deep an application of ink, is frequently unnecessarily large and can diffuse around the application puncture site. The result is a large unsightly tattoo that is permanent. Frequently, such a tattoo is an all too visible reminder of an extremely unpleasant period in a patient's life. With pre-loaded elastic drive, limitation to a single use, and self-contained ink reservoir, the disclosed embodiments meet the needs of simplicity, cost effectiveness, and sanitation that the prior art does not adequately address. The pre-calibrated force applied by the elastic drive keeps the needle penetration shallow and painless.

In addition to the mechanical improvements the current invention makes over prior art and methods, the described embodiments make further improvements through the selection of ink. Inks used in the prior art are difficult to be seen in several situations. If the receiver of the medical tattoo has dark skin, the location for the medical tattoo coincides with a previously acquired art tattoo, or if a birth mark or other natural skin coloration coincides with the target area, the medical tattoo will lack the desired visibility. In one of its embodiments, the current invention utilizes an ink that is invisible under normal lighting conditions, but illuminates under ultraviolet light. This provides a medical tattoo that is visible only when it is desired for it to be visible.

### DESCRIPTION OF PRIOR ART

U.S. Pat. No. 2,699,784 by G. M. Krayl is for an apparatus for piercing the skin. It consists of a long tubular body being hollow on the inside and having caps on each end. In the central portion of the hollow body are a spring, a plunger with a striking face that is propelled by the spring, and a piercing needle which is struck by the plunger. Each end cap has an aperture through it. At one end, the plunger extends beyond the tubular body and has a cap attached to it. The cap is used to draw back the plunger and cock the spring and plunger. Along the side of the body of the apparatus is attached a latch which protrudes through an opening in the side of the body to catch the striking member. This latch retains the striking member in a cocked position and is tripped to release the striking member to strike the needle. The needle extends through the aperture through the other end cap to pierce the skin. The needle pierces through the skin for blood samples, etc.

U.S. Pat. No. 5,540,709 by Ramel is a lancing device for piercing the skin. The overall shape of the device is cylindrical. At a closed end of the device is a seat where a spring is seated as well as the carrier for the lancing needle. This end also has hooked tabs which hold the carrier of the needle in a cocked position with the spring compressed behind the needle carrier. On the opposite end and extending out from the end of the cylindrical body is the trigger device. The trigger itself is a hollow cylinder and its inner diameter accommodates the carrier of the needle when the carrier is propelled by the spring. In use, the trigger, which extends past the end of the overall apparatus body, is driven back when the apparatus is pressed against the skin. When it is driven back, the trigger's interior end presses against the hooked tabs that restrain the needle carrier and spring. This spreads the hooked tabs and releases the needle carrier to be driven by the spring to the exposed end of the trigger. The needle extends through the exposed end of the trigger and pierces the skin. With this configuration, the apparatus achieves a lancing of the skin with a device that may be triggered by merely pressing it against the skin.

U.S. Pat. No. 6,322,574 by Lloyd, et al. is a disposable lancet which uses a leaf spring to hold the lancet in a retracted position as opposed to using a spring to drive the lancet. The overall structure of the device is a cross consisting of two crossing members. One of the crossing members is larger than the other with the shorter crossing member being located closer to one end of the larger crossing member. These crossing members are perpendicular. The lance of the apparatus is located internal to the larger crossing member and near the end of the longer section. A leaf spring located internal of the shorter crossing member biases the lance to a normal retracted position. A button located at the short end of the larger crossing member is capable of driving the lance to extend it out of the long end of the larger crossing member. In operation, the lancet device is placed in contact with the skin at the long end of the longer crossing member and fingers are used to hold the device with the shorter crossing members. The thumb is used to press the button to extend the lancet and overcome the resistance of the leaf spring. The lancet extends and pierces the skin.

U.S. Pat. No. 3,208,452 by J. J. Stern is a device for lancing the skin or otherwise scarifying it for the application of medicine to the scarified skin. It comprises an overall cylindrical body, with the scarifying needles at one end. A button or handle extends from the other end. From the sides of the cylindrical body, extend two levers which may be used to anchor the fingers with a thumb being able to be pressed on the handle or button extended from one end of the cylindrical body. Within the cylindrical body, the cylindrical carrier of the needles is restrained by balls which rest on a annular shoulder on the inner diameter of the apparatus body and which engage notches in the cylinder. A shaft attached to the button and/or handle passes through the body on down to the needle carrier. The shaft advances when the button is pushed and begins to compress a spring situated against the needle carrier. As the shaft advances, notches in the shaft come into alignment with the notches in the carrier which allows the balls to disengage from the annular shoulder in the inner diameter of the body cylinder. This releases the shaft and carrier to be driven by the compressed spring and pushes the scarifying needles to extend from the apparatus and mark, puncture, or otherwise abrade the surface of the skin.

U.S. Pat. No. 6,685,719, by Matera Jr., discloses an apparatus for dispensing tattoo ink and a method of using the same. The apparatus includes an ink cartridge, a leading cap, and a trailing cap. The ink cartridge has a slight conical shape with a small tube leading from the main chamber of the ink cartridge to the smaller end of the ink cartridge which is the ink dispensing tip. The leading cap covers this dispensing tip of the ink cartridge, while the trailing cap is used to seal the larger end of the ink cartridge after it has been filled. An ultrasonic weld is the contemplated method of sealing the trailing cap to the ink cartridge.

The apparatus is used by removing the leading cap and allowing ink to flow from the reservoir in the ink cartridge to the tip of the ink cartridge. The ink cartridge may be squeezed to cause ink to flow, or a gravity feed may also be used. In addition to the ink cartridge, the leading cap, and the trailing cap, a needle having a lumen may also be used with the apparatus. The needle would fit over the leading edge of the ink cartridge where the ink is dispensed and the lumen passes along its length. Squeezing the cartridge would cause ink to travel from the cartridge down through lumen in the needle to its tip, where it could be used to mark the skin for whatever purposed desired. This method introduces a relatively large quantity of ink below the skin. In the alternative, the ink may merely be applied to the skin, and a needle used after the fact to conduct the ink into the skin. Here again, this method will likely result in actual puncturing of the skin and excessive ink being deposited in the skin. There is also a patent application Ser. No. 10/757,009, pending from the same initial application as the issued patent, U.S. Pat. No. 6,685,719.

U.S. Pat. No. 5,810,862, by Pilmanis, uses several needles clustered together as the central feature of the invention. The needles are arranged so that their pointed ends form a plane at a sixty degree angle to the long dimension of the needles. They are held in this arrangement by a small mass of solidified glue.

In addition to the bundle of needles, the device has a holder, an elongated tube and a cap. The holder is the part that actually holds the bundle of needles and it screws onto the end of the elongated tube. A resilient biasing device placed within the long tube presses against the back of the needles and keeps them in place in the holder. The cap may be placed over the holder to cover the needles or removed and placed over the end of the long tube. In an alternative embodiment, rather than a resilient member pressing at the back of the needles, glue may be used to hold the bundle of needles in place. It is envisioned that with the embodiment that uses an elastic member to hold the needles in place, the elastic member and the needles would be able to be sold separately as a replacement set for the apparatus. The invention in Pilmanis does not include a reservoir for pigment and appears to rely upon application of the pigment directly to the skin and perforation of the skin with the device claimed in Pilmanis to achieve the tattoo desired or the infusion of the pigment into the skin.

U.S. Pat. No. 4,665,912 by Burton discloses and claims a device for transferring pigment into the skin that is very similar to a common ballpoint pen. The pen-like structure has a small ink reservoir at the end where the ballpoint would normally extend. This reservoir is interior to the body. Within that reservoir, in a normally retracted position, a needle is held, which may be advanced and retracted in a means similar to the pen and ink cartridge of a ballpoint pen. The opening at the end of the body of this device is sized to fit closely about the needle when the needle is extended but there is enough of a gap that ink may still flow due to capillary action. The device is put into use by extending the needle and allowing this capillary flow to bring a bead of ink to the end of the opening where the needle is extended. The ink is deposited to the surface, and then the skin is perforated by the needle repeatedly to allow the ink to transfer through and into the skin. Alternatively, the needle may be retracted and the ink then flows to the tip of the invention and is applied to the skin. Then the needle is extended, and again, used to perforate the skin. Generally this device is intended to be used to apply a more permanent eyeliner for makeup purposes. Internal to the device, above the area where the ink reservoir is located, seals may be put in place so that the ink may not traverse up higher into the device where springs and other small mechanisms are located which allow the needle to be extended and retracted.

U.S. Pat. No. 6,626,927 B1 by Koplen is directed to a tattooing system that is intended to be easier to clean. The system comprises a tube member which has a first end for receiving the drive unit, a threaded end for receiving a replaceable ink reservoir, and a central channel for receiving a tattoo needle which extends from the drive unit, through the tube member, and on through the replaceable reservoir tip to extend out where the ink will be applied. The tip member having the reservoir also has threads on its internal surface matched to the threads on the tip of the tube member. The tip and reservoir are placed on the end of the tube member by engaging these threads and the surfaces between the two members are adapted to seal once the tip is placed on the tube member. To use the device, the drive unit is engaged, and the needle is driven with the reciprocating motion to extend out beyond the tip, alternatively retracting within the reservoir. In so doing, the needle carries ink upon its surface, so that when it is held proximate to the skin, the needle pierces the skin and takes ink with it into the skin. The results are multiple piercings of the skin to a shallow, desired depth with these piercings being infused with indelible ink. The intention of this invention is to provide an easily cleaned device that may be reused with a lower incidence of contamination.

### SUMMARY OF THE INVENTION

The apparatus of the present invention relates to applying small medical tattoos for the purpose of accurately locating recurring medical treatments. In the described embodiments, the apparatus is capable of producing only a single tattoo after which it is discarded for sanitary purposes. Also for sanitary purposes, the apparatus may carry its own ink reservoir through which the tattooing needle passes to transfer ink to the recipient's skin. This avoids the use of a common ink supply for several different persons. The apparatus has a hollow body, and in some embodiments of the invention, some portion of that hollow body contains an ink reservoir. Also within the hollow body, a tattoo needle is located. An elastic drive member is pre-loaded, temporarily anchored, and ready to drive the tattoo needle. The device has a means for overcoming the temporary anchor of the elastic drive member to release the drive member to drive the tattoo needle through the reservoir and apply the tattoo. Alternatively, ink from an ink supply separate from the claimed device could be applied to the skin and then the device used to mark the skin, or the ink reservoir could be located on the device and the ink applied to the skin before marking with the device without requiring the needle to pass through the reservoir.

In this respect, before explaining at least one embodiment of the invention in detail, it is to be understood that the invention is not limited in its application to the details of construction and to the arrangement of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

Accordingly those skilled in the art will appreciate that the conception upon which this invention is based may readily be utilized as a basis for the design of other structures, methods and systems for carrying out the several purposes of the present invention. It is important, therefore, that the claims be regarded as including such equivalent constructions insofar as they do not depart from the spirit of the present invention.

Furthermore, the purpose of the foregoing Abstract is to enable the U.S. Patent and Trademark Office and the public generally, and especially including the practitioners in the art who are not familiar with patents or legal terms or phraseology, to determine quickly from a cursory inspection, the nature and essence of the technical disclosure of the application. The Abstract is neither intended to define the invention of the application, nor is it intended to be limiting to the scope of the invention in any way.

### BRIEF DESCRIPTION OF THE DRAWINGS

Additional utility and features of the invention will become more fully apparent to those skilled in the art by reference to the following drawings, which illustrate the primary features of several embodiments including a preferred embodiment.

FIG. 1 shows a cross section of a first embodiment of the invention before being used.

FIG. 2 shows the unmoving components of a first embodiment in an exploded arrangement.

FIG. 3 shows the moving components of a first embodiment in an exploded arrangement.

FIG. 4 is an enlarged side view of the needle cap of the needle assembly of a first embodiment.

FIG. 5 is an enlarged top view of the needle cap of the needle assembly of a first embodiment.

FIG. 6 is a cross section view of a first embodiment in the plane indicated in FIG. 1 showing the needle cap held by a catch.

FIG. 7 is an enlarged view of the piercing end of the needle.

FIG. 8 shows a cross section of the first embodiment with the needle extended to apply the tattoo.

FIG. 9 shows a cross section of a second embodiment of the invention before being used.

FIG. 10 shows a cross section of the second embodiment of FIG. 9 with the needle extended to apply the tattoo.

FIG. 11 shows the moving parts of the second embodiment of FIG. 9 in an exploded arrangement.

FIG. 12 shows a cross section of the body end and button mechanism of the second embodiment of FIG. 9.

FIG. 13 is a cross section of a third embodiment that starts with a drive spring in tension before being used.

FIG. 14 is a cross section of the third embodiment that starts with a drive spring in tension with the needle extended to apply the tattoo.

FIG. 15 is a cross section of a fourth embodiment that comprises features of both the first and second embodiments.

### DETAILED DESCRIPTION OF THE PREFERRED EMBODIMENT

The detailed description below is for multiple embodiments and is intended to explain the current invention. It is to be understood that a variety of other arrangements are also possible without departing from the spirit and scope of the invention. Generally, the embodiments include; a hollow body, a needle assembly temporarily anchored within the hollow body, means for overcoming the temporary anchoring of the needle assembly, a first elastic member that is pre-loaded to drive the needle assembly when the temporary anchoring means is overcome, and in some embodiments, an ink reservoir and a second elastic member to retract the needle assembly after the tattoo has been created.

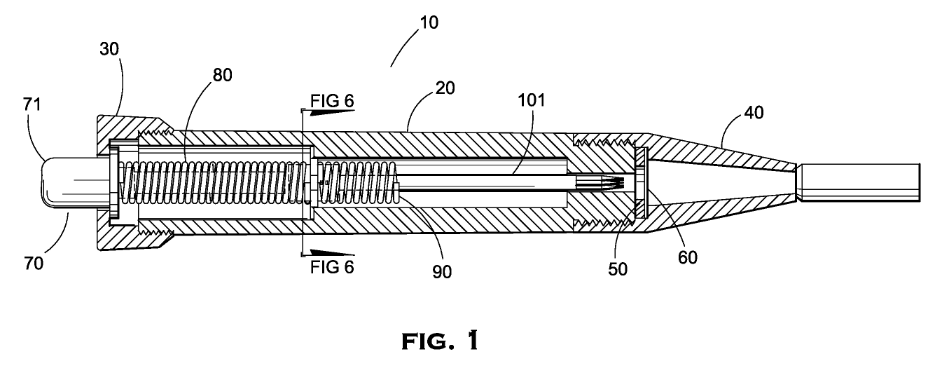

xxxFIG. 1 shows a cross section of a first embodiment of the mapping device 10 before being used. The structural components of the device 10 comprise a hollow body 20, an end cap 30, reservoir end 40 as well as needle guide 50 and diaphragm 60 located between body 20 and reservoir end 40. The moving parts of mapping device 10 are visible within the hollow space viewable in the cross section view FIG. 1. These moving parts include button assembly 70, of which button 71 is most visible, drive spring 80 or first elastic member, rebound spring 90 or second elastic member, and a needle assembly, of which needle 101 is most visible.

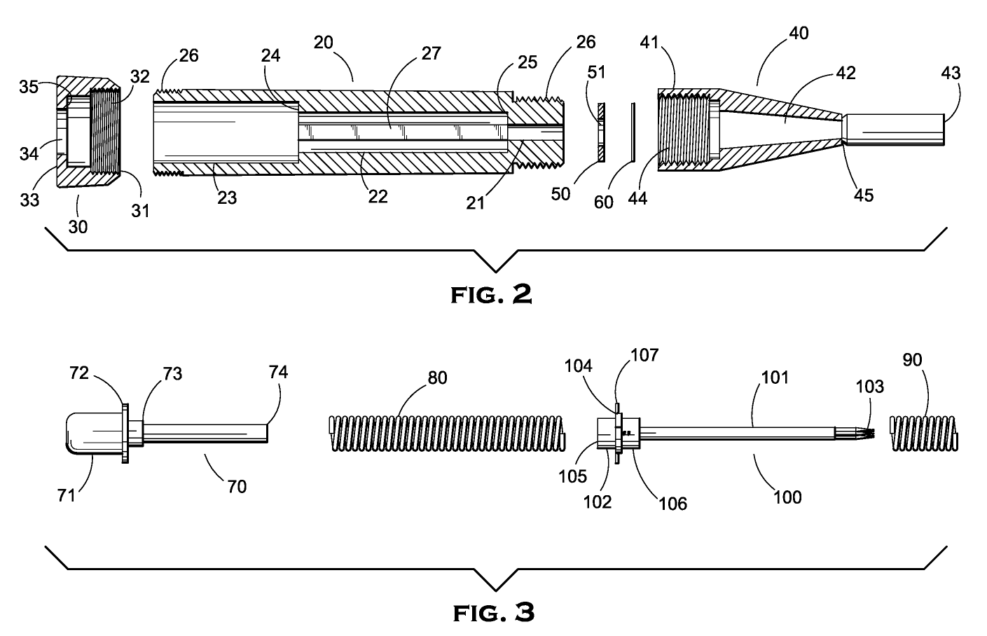

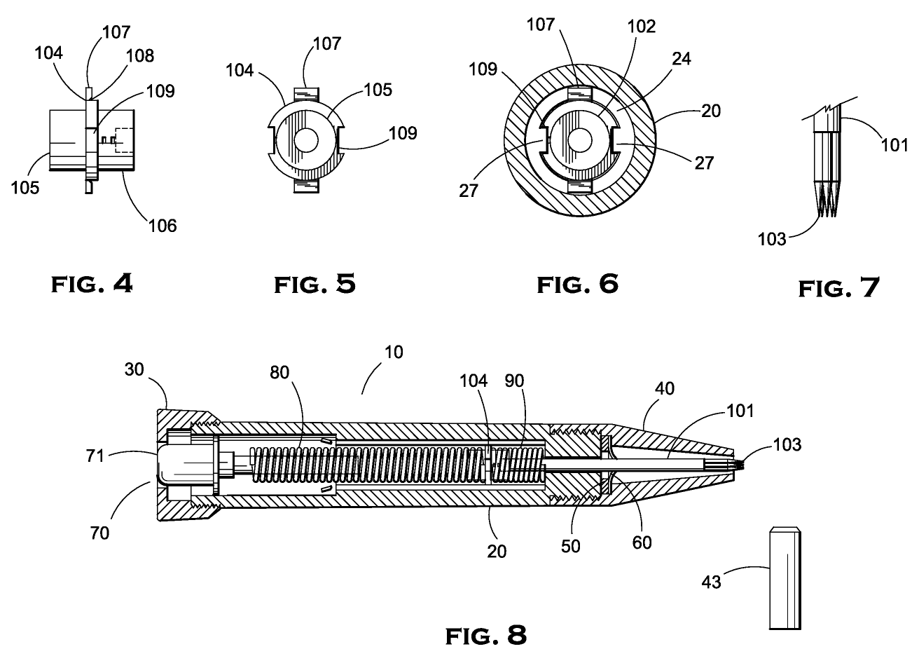

Referring now to FIG. 2 which shows the unmoving components in an exploded arrangement, body 20 will be considered first. Within hollow body 20 there are three sections. On the same end of body 20 as reservoir end 40 is needle tube 21. The inner diameter of body 20 in the needle tube 21 section is sized to accommodate a slip fit with needle 101 as seen in previous FIG. 1. Needle tube 21 is the shortest section within body 20. Spring guide 22 is the central section of body 20 and its internal diameter is sized to provide a slip fit for rebound spring 90, needle cap 102, and drive spring 80. Needle cap 102 may be seen more clearly in FIG. 3, FIG. 4, and FIG. 5, and it may be seen in body 20 in FIG. 8, and FIG. 1 and FIG. 6 as located by seat rim 104. The spring guide 22 section of body 20 is the longest of the three sections, but its inner diameter is sized between the inner diameter of the other two sections. The final section within body 20 runs from spring guide 22 to the end of body 20 that is covered by end cap 30. This section, or button chamber 23, has a larger inner diameter than its neighboring section in body 20, spring guide 22.

The differing inner diameters between the three internal sections of body 20 requires some transition between the sections. In the first embodiment, these transitions are perpendicular to the axis of body 20. The transition between button chamber 23 and spring guide 22 produces catch 24 which is used to temporarily anchor needle assembly 100. The transition between spring guide 22 and needle tube 21 is larger than catch 24 and its proximity to the end of body 20 causes it to function as a shaft bottom 25. At each end of body 20, external threads 26 on its outer diameter provide for coupling with end cap 30 and reservoir end 40.

Still referring to FIG. 2, separated end cap 30 may be seen more clearly. End cap 30 is cylindrical and in the form of a slightly elongated hoop with one end partially closed. On the inside rim 31 of end cap 30 are internal threads 32 which couple with external threads 26 of body 20 to hold the two together. Opposite the internally threaded inside rim 31 is top 33 of end cap 30, through which button aperture 34 passes. As can be seen in FIG. 1, button 71 of button assembly 70 passes through button aperture 34. The part of top 33 not precluded by button aperture 34 forms button seat 35 internal of end cap 30.

Referring again to FIG. 2, separated reservoir end 40 may also be seen more clearly. Reservoir end 40 has three sections; coupling section 41, reservoir 42, and end tab 43. Coupling section 41 and reservoir 42 are hollow sections. Coupling section 41 has an inner diameter and internal threads 44 which couple with external threads 26 on the outer diameter of needle tube 21 of body 20. Reservoir 42 is generally conical on its internal and external surface and tapers from a larger cross section at the junction between coupling section 41 and reservoir 42 to a smaller cross section at the junction of reservoir 42 and end tab 43. End tab 43 is solid, but it is detachable from reservoir 42 at the reduced end of reservoir 42. A further reduced neck 45 at the junction between reservoir 42 and end tab 43 facilitates the removal of end tab 43.

Referring still to the exploded view of FIG. 2, needle guide 50 and diaphragm 60 are located between body 20 and reservoir end 40 with needle guide 50 placed against main shaft 20. Both needle guide 50 and diaphragm 60 are thin disks, but needle guide 50 is thicker than diaphragm 60 and has a guide aperture 51 through it. Diaphragm 60 serves to seal the large end of reservoir 42 in reservoir end 40.

Referring now to FIG. 3, the moving elements of the first embodiment can be seen removed from body 20 and in an exploded arrangement. At the left, button assembly 70 is shown. At the left end of button assembly 70 in FIG. 3 is button 71, which may be seen protruding in FIG. 1 from end cap 30 of device 10. Button assembly 70 is generally cylindrical in shape but has several different diameters along the length of its axis. After button 71 comes button rim 72 which is how button assembly 70 is maintained in its position. Next to that is button hub 73 which is sized to fit within drive spring 80 which is shown next to button assembly 70 in FIG. 3. Punch 74 extends from button hub 73 and is the longest section of button assembly 70. When device 10 is fully assembled, punch 74 is directed towards needle assembly 100 and is in close proximity to it in the assembled position.

Next to button assembly 70 is drive spring 80 which is a compression spring. Drive spring 80 fits over punch 74 and button hub 73 of button assembly 70. Its other end presses up against needle cap 102 of needle assembly 100 and is placed in compression holding each of those elements in their positions. Drive spring 80 is in a pre-loaded state while holding these elements in position.

As just stated, drive spring 80 interacts with needle assembly 100 at its right in FIG. 3. Needle assembly 100 comprises two major components. They are needle 101 and needle cap 102. Needle 101 has a piercing end 103 and its other, non-piercing end is covered by needle cap 102. Central on needle cap 102 is seat rim 104, either side of which provides a spring seat. On the left side of needle cap 102 is drive hub 105 which fits within drive spring 80. That side of seat rim 104, therefore, provides a seat for drive spring 80. On the opposing side of needle cap 102 is rebound hub 106 which fits into rebound spring 90 and that side of seat rim 104 is the seat for rebound spring 90. Extending from seat rim 104 perpendicular to the length of needle assembly 100 are anchoring tabs 107. In assembled location, anchoring tabs 107 rest upon catch 24 of body 20 shown in FIG. 2 and provide a temporary anchoring means for needle assembly 100.

FIG. 4 shows an enlarged detail of needle cap 102. Looking at FIG. 4, it can easily be seen that where anchoring tab 107 joins seat rim 104, anchoring tab 107 is reduced to create fracture notch 108. Fracture notch 108 ensures that anchoring tabs 107 break off right at seat rim 104 and thus allow needle assembly 100 to pass easily through needle tube 21 of body 20.

FIG. 5 shows needle cap 102 from the drive hub 105 end of needle cap 102, while FIG. 6 shows a cross section view in the plane indicated in FIG. 1 showing anchoring tabs 107 of needle cap 102 resting on catch 24. Guide notches 109 in seat rim 104 ride along guide rails 27 as needle assembly 100 is driven through needle tube 21 of body 20. Guide notches 109 and guide rails 27 keep needle assembly 100 aligned as it passes through body 20. A guide rail 27 may be seen in FIG. 2 with the working elements of the tattooing device removed. As can be seen in FIG. 5 and FIG. 6, in the first embodiment, anchoring tabs 107 are positioned ninety degrees around from guide notches 109 and guide rails 27.

FIG. 7 shows piercing end 103 of needle 101 in more detail. Piercing end 103 may be comprised of several smaller needles affixed to needle 101. These may be soldered along their lengths or otherwise fixed together as well as fixed to the shaft of needle 101. This provides a means of applying a tattoo having more than a single dot, which will, therefore, be more visible.

Having thus described elements of the mapping device 10, reference will be made to FIG. 1 once again, as well as FIG. 8, to discuss the workings of the device 10. Looking at FIG. 1, the components of device 10 may be seen in assembly and in a pre-loaded state. Button assembly 70 is located in the left end of device 10 and it is held in place by drive spring 80 which is in compression between button assembly 70 and needle assembly 100. This also holds needle assembly 100 in place at catch 24 within body 20. To use the device to apply a tattoo, the end tab 43 is broken off of reservoir end 40 and the device is placed upon the skin at the location where the tattoo is desired. Button 71 is depressed driving punch 74 against needle cap 102 until anchoring tabs 107 are overcome and break. At that time, drive spring 80, which is under compression, pushes needle assembly 100 through needle tube 21, and piercing end 103 of needle 101 punctures diaphragm 60, passes through reservoir end 40, extends out of device 10, perforates the skin with ink present, and, thereby, creates a tattoo in the skin. FIG. 8 shows the apparatus after anchoring tabs 107 have been overcome, diaphragm 60 has been punctured, and needle 101 is extended from reservoir end 40 to pierce the skin and create a tattoo. Subsequently, rebound spring 90 will retract needle 101 so that piercing end 103 is retracted into reservoir end 40.

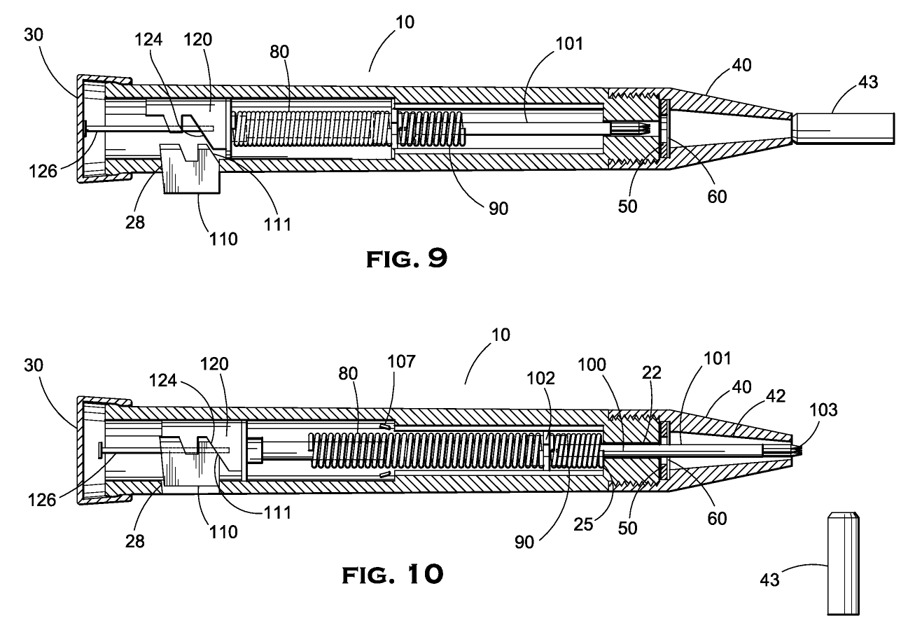

FIGS. 9 and 10 show an alternative embodiment of device 10. Where possible and appropriate, the same numbers will be used for the same features. The two embodiments are essentially the same from the drive spring 80 to end tab 43 on reservoir end 40. The essential differences between the first embodiment and the second embodiment is in the method of actuating punch 74 that overcomes and breaks off anchoring tabs 107 and releases needle assembly 100. The mechanism used in this embodiment is a cam and cam follower. In this case, the cam is a very simple wedge arrangement. As can be seen in FIG. 9, an aperture 28 in the side of body 20 allows the cam button 110 of this mechanism to protrude from the side of body 20. The sides of cam button 110 taper from large to small as they pass from the inside of device 10 to the outside so that cam button 110 is maintained in its position before device 10 is used.

Referring now to FIG. 11, the button mechanism is shown outside of body 20 and in an exploded view. New numbering will be used for the new pieces in this assembly. However, for the drive spring 80 and needle assembly 100, no new numbers are needed.

Referring to FIG. 11, cam button 110 is sized and shaped to fit aperture 28 as shown in FIG. 9 and FIG. 10. Cam button 110 also has extending into the interior of body 20 a cam surface 111, oblique to the axis of body 20. In the second embodiment as shown, cam surface 111 is flat and does not have any curvature.

Also in FIG. 11, punch assembly 120 is shown, which is in some parts the same as button assembly 70 of the prior embodiment. The similarities include punch 121, punch hub 122, and punch rim 123. Drive spring 80 fits over punch 121 and punch hub 122 just as drive spring 80 fits over punch 74 and button hub 73 on the previous embodiment. Punch rim 123 fits closely within button chamber 23 as before and keeps punch assembly 120 aligned. To the left of punch rim 123 are follower surface 124 and guide surface 125. Follower surface 124 is oblique to the axis of body 20 and is at an angle to match cam surface 111 of cam button 110. Guide surface 125 is shaped to fit the contour of body 20. An additional element of this embodiment is spacer pin 126. Spacer pin 126 nests into a cavity in punch assembly 120 at one end, while its other end braces at the closed end of body 20. Spacer pin 126 keeps drive spring 80 in a pre-loaded state and keeps the assembly in place.

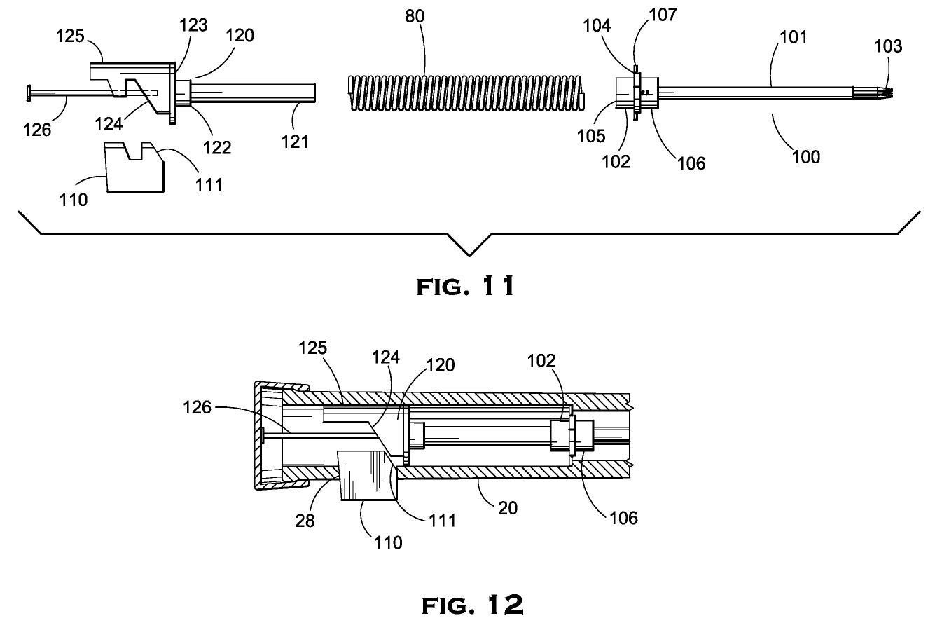

FIG. 12 shows the portion of the second embodiment that is different from the first with drive spring 80 of other figures removed. This shows the interaction of the pieces more clearly. Also cam button 110 is simplified to more clearly show the cam surfaces and wedging effect. Cam button 110 extends through aperture 28 to where cam surface 111 is in parallel with follower surface 124 of punch assembly 120. Spacer pin 126 maintains the location of punch assembly 120 and biases it towards needle cap 102 of needle assembly 100. Guide surface 125 maintains contact with the inside of body 20. Referring now back to FIGS. 9 and 10 showing the full assembly, where FIG. 9 is before use of device 10 and FIG. 10 is after use, it can be seen in FIG. 10 that cam button 110 has advanced into the interior of body 20, cam surface 111 has made contact with follower surface 124, and cam surface 111 has driven punch assembly 120 down to press needle cap 102 of needle assembly 100. Anchoring tabs 107 on needle assembly 100 have broken off and needle assembly 100 has passed down through spring guide 22 and piercing end 103 of needle assembly 100 has passed through diaphragm 60, and reservoir 42 of reservoir end 40 to extend beyond the end of reservoir end 40. Rebound spring 90 is now in contact with shaft bottom 25 and the working parts of device 10 have reached an extended position where the extended needle 101 would pierce the skin that it was placed near and create a tattoo from the ink present from reservoir 42. Subsequently, rebound spring 90 will move the assembly back to a new static position with piercing end 103 retracted back into reservoir 42.

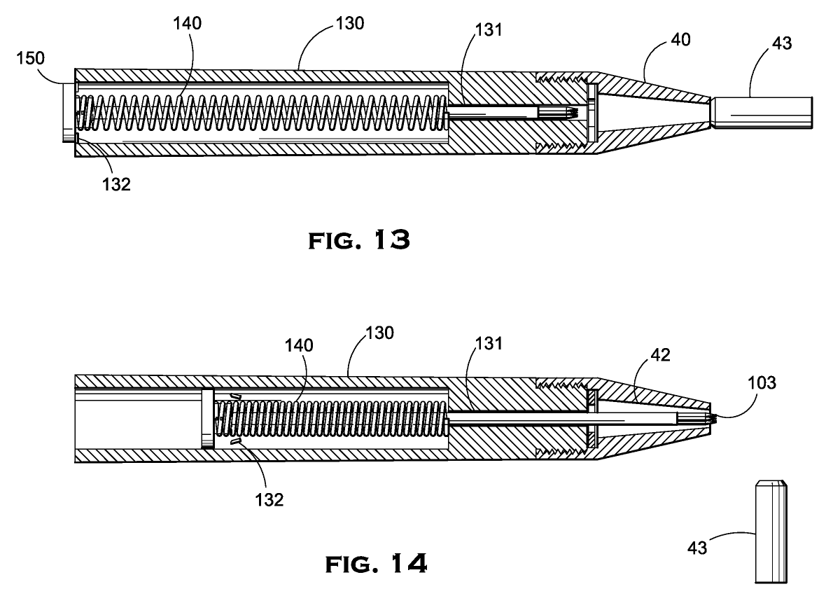

FIG. 13 and FIG. 14 show an alternative embodiment of the tattoo device which uses an elastic member in tension for the motive force. Body 130 of this embodiment has a needle tube 131 at one end as in the prior discussed embodiments, but the balance of its internal section is single diameter with the exception of the end which retains the button assembly and needle assembly which are combined in this embodiment. This end is open with the exception of the restriction caused by anchoring tabs 132 which are located on body 130 and protrude into the internal section of body 130. These anchoring tabs 132 temporarily restrain button and needle assembly 150. Both button and needle assembly 150 and body 130 have anchor points on them which allow an elastic member to be fixed to them. Pull spring 140 has one end anchored within body 130 at the end where needle tube 131 is located while its other end is anchored to button and needle assembly 150. Initially, pull spring 140 is pre-loaded in tension. Reservoir end 40 is constructed similar to reservoir end 40 in the previous embodiments and is located over the end of body 130 where needle tube 131 is located. To use the device, tab end 43 is broken off of reservoir end 40 and device 10 is placed near the skin. Pressing the exposed button of button and needle assembly 150 protruding from the end of body 130 causes anchoring tabs 132 to break and button and needle assembly 150 is released and pull spring 140 pulls button and needle assembly 150 through the interior of body 130. The needle extends through needle tube 131 and reservoir end 40 to place a tattoo on the skin. The unloaded state of pull spring 140 is such that its coils do not pull in flat against each other, but rather there are spaces between the coils and pull spring 140 also acts as a rebound string. After the tattoo is created, pull spring 140 moves button and needle assembly 150 back into body 130 with piercing end 103 retracted into reservoir 42. This embodiment uses a spring, or elastic member, pre-loaded in tension for its motive force compared to the springs in compression in the prior embodiments. This configuration would even allow an elastic band to provide the motive force for driving the needle with an elastic diaphragm providing the means of retracting the piercing end the small distance required to move the needle tip back into the reservoir.

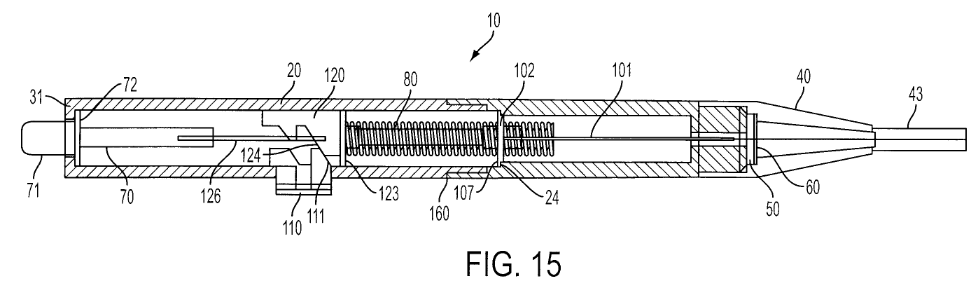

FIG. 15. illustrates an embodiment of the invention that comprises features of both the embodiments illustrated in FIGS. 1 and 2, and FIGS. 9 and 10 and associated figures. The features combined in this embodiment allows the mark to be applied by pushes either a side button or an end button. This gives more versatility in how the device is held when a mark is applied to different locations on the body.

The assembly comprising needle 101 and needle cap 102 is restrained at anchoring tabs 107 by catch 24. Drive spring 80 is compressed between needle cap 102 and punch rim 123 of punch assembly 120. As with the embodiment shown in FIGS. 9 and 10 and associated figures, punch assembly 120 has a follower surface 124 that is contacted by the cam surface 111 on cam button 110 which protrudes through the side of body 20. In this embodiment, punch assembly 120 is held in place by spacer pin 126 which partially inserts into button assembly 70. Much as in the first embodiment discussed, button 71 on button assembly 70 protrudes out the end of device 10, but in that first embodiment shown in FIG. 1, end cap 30 provides the stop for button 71. In the present embodiment, body 20 is constructed in two segments, and the end through which button 71 protrudes has an inside rim 31, which button rim 72 presses against. Button rim 72 maintains button assembly 70 in its place against compressed drive spring 80 which is pushing against punch rim 123 and then through spacer pin 126 on into button assembly 70. The two segments of body 20 are connected at joint 160. Whether an end cap construction is used, or whether body 20 is constructed in two pieces with an inside rim 31 built into the appropriate piece to restrain button 71, is a matter of manufacturing preference. The different embodiments shown in the drawings having different methods of assembly should illustrate the several ways of accomplishing the invention and should not be regarded as limiting the scope of the invention in any way.

Pressing cam button 110 causes cam surface 111 to engage follower surface 124 which moves punch assembly 120 until anchoring tabs 107 break, releasing the needle assembly to be driven by drive spring 80 and apply the mark. Alternatively, pressing button 71 drives spacer pin 126 into punch assembly 120, again moving punch assembly 120 until anchoring tabs 107 break and release the needle assembly. This gives more versatility to using the device.

Each of the embodiments discussed above may use different inks. One embodiment uses an ink that is normally not visible, but becomes visible when exposed to light of predominantly narrow spectrum such as ultraviolet light. This allows the tattoo to remain invisible in normal light but visible when needed.

Having provided detailed descriptions of several embodiments, it should be noted that there are several means to vary the specific sizing and arrangement but still accomplish the overall goal of the invention. It should be obvious from this that there are numerous embodiments subsumed in the present invention and the scope of this invention should not be limited by the discussion of the embodiments above. Neither the specification, nor the abstract, should be taken as an exhaustive illustration of the invention.

For example, end tab 43 common to several embodiments serves as a removable seal for reservoir end 40. This function can be served by several known means such as a plug, or thin membrane. While not illustrated, these methods would also seal the reservoir end for purposes of the instant invention. Similarly, needle guide 50 and diaphragm 60 could easily be combined by placing a laminate on a substrate. The attributes of sealing and guiding would still be accomplished with slight changes in the arrangements.

As to the shape of the body of the device being a shaft as in body 20, this is merely a result of one method of fabrication and should not be regarded as a defining limitation. Should another method of fabrication, such as a clam shell assembly be selected, the possible shapes for the body of the device would be nearly unlimited.

Interrelated with the shape of the body of the device is the elastic drive means. The previously described embodiments of the invention, utilize elastic drive means capable of operating in a linear fashion. Differing the structure of the body would allow other elastic drive means such as cantilever springs, leaf springs, or elastic bands acting transverse to their length. Also, interrelated with the method of fabricating the body and its shape, is the particular means of guiding the needle.

As discussed previously, the temporary anchoring means for the needle assembly and pre-loaded elastic drive may also vary. If break-away tabs are used, the tabs themselves may be located on the needle assembly or on the device body. Break-away tabs are not the only possible temporary anchoring means. The temporary anchoring means may comprise flexible pegs engaging recesses. Also an interference fit between the device body and the needle assembly which may be overcome by a user but not by the elastic drive means is a possible temporary anchoring means, along with various latching and tripping means widely known. For overcoming the temporary anchoring means, a linear punch and a cam driven punch have already been discussed. There are numerous means such as levers and various mechanical trips which could also be used and be within the scope of the current invention.

I claim:

1. A medical marking device, comprising;
a) a hollow body having a first opening and a second opening;
b) a marking needle located within said hollow body, said marking needle having a piercing end and a non-piercing end with its piercing end directed toward said first opening of said hollow body;
c) a needle cap, said needle cap being located on said non-piercing end of said marking needle;
d) temporary anchoring means whereby said marking needle and said needle cap are held in an initial position;
e) a first elastic member having a first end and a second end, said first end of said first elastic member being operatively associated with said marking needle at said needle cap, and said first elastic member being pre-loaded by the location of said second end;
f) at least one means for overcoming or releasing said temporary anchoring means.

2. The medical marking device of claim 1, further comprising;
an ink reservoir, said ink reservoir being located at said first opening of said hollow body and having means for said piercing end of said marking needle to extend through said ink reservoir out of said medical marking device.

3. The medical marking device of claim 1, wherein said temporary anchoring means comprises;
a) at least one breakable tab on said needle cap, and;
b) a catch on the inside surface of said hollow body, wherein;
c) said breakable tab rests upon said catch.

4. The medical marking device of claim 1, wherein said at least one means for overcoming said temporary anchoring means comprises;
a) a button assembly slidably located within said hollow body, said button assembly having a button end and a punch end, wherein;
b) said button end of said button assembly protrudes from said second opening of said hollow body, and;
c) said punch end of said button assembly is in proximity to said needle cap.

5. The medical marking device of claim 4, wherein;
a retaining cap fits over said second opening of said hollow body to retain said button assembly.

6. The medical marking device of claim 1, wherein;
said first elastic member is a compression spring having said second end of said first elastic member located proximate to said second opening of said hollow body.

7. The medical marking device of claim 6, further comprising;
a) a second elastic member positioned within said hollow body, said second elastic member having a first end and a second end, wherein;
b) said first end of said second elastic member is operatively associated with said needle cap on the side opposite said first elastic member, and wherein;
c) said second end of said second elastic member is directed toward said first opening of said hollow body.

8. The medical marking device of claim 1, wherein;
said first elastic member is a tension spring having said second end of said first elastic member located proximate to said first opening of said hollow body.

9. The medical marking device of claim 2, wherein said ink reservoir comprises;
a) a hollow tip, having a large opening and a small opening in line with each other, and;
b) ink within said hollow tip, wherein;
c) said large opening of said hollow tip fits onto said first opening of said hollow body.

10. The medical marking device of claim 9, further comprising;
a breakable tab located over said small opening of said ink reservoir, said breakable tab being removable when said medical marking device is to be used.

11. The medical marking device of claim 9, wherein;
a seal covers said large opening, said seal being pierceable by said marking needle.

12. The medical marking device of claim 1, wherein said at least one means for overcoming said temporary anchoring means comprises;
a) an aperture through the wall of said hollow body, said aperture being located between said needle cap and said second opening of said hollow body;
b) a cam assembly slidably located within said hollow body, said cam assembly having a cam end and a punch end, said punch end being directed toward said needle cap and said cam end having a surface oblique to the length of said marking needle, said oblique surface being directed toward said second opening of said hollow body and proximate to said aperture;
c) a spacer pin located within said hollow body and maintaining a distance between said inclined surface and said second opening of said hollow body;
d) a cam button sized to fit through said aperture in said hollow body, said cam button having a surface oblique to the length of said marking needle in a manor nearly parallel to said oblique surface of said cam assembly and said cam button protruding from said aperture in said hollow body.

13. The medical marking device of claim 1, wherein said at least one means for overcoming said temporary anchoring means comprises;
a first means for overcoming said temporary anchoring means, and a second means for overcoming said temporary anchoring means;
said first means for overcoming said temporary anchoring means comprising;
a) a button assembly slidably located within said hollow body, said button assembly having a button end and a punch end, wherein;
b) said button end of said button assembly protrudes from said second opening of said hollow body, and;
c) said punch end of said button assembly is in proximity to said needle cap, and;
said second means for overcoming said temporary anchoring means comprising;
d) an aperture through the wall of said hollow body, said aperture being located between said needle cap and said second opening of said hollow body;
e) a cam assembly slidably located within said hollow body, said cam assembly having a cam end and a punch end, said punch end being directed toward said needle cap and said cam end having a surface oblique to the length of said marking needle, said oblique surface being directed toward said second opening of said hollow body and proximate to said aperture;
f) a spacer pin located within said hollow body and maintaining a distance between said inclined surface and said second opening of said hollow body;
g) a cam button sized to fit through said aperture in said hollow body, said cam button having a surface oblique to the length of said marking needle in a manor nearly parallel to said oblique surface of said cam assembly and said cam button protruding from said aperture in said hollow body.
